(19) United States
(12) Patent Application Publication
(10) Pub. No.: US 2004/0087633 A1
(43) Pub. Date: May 6, 2004

(54) Methylidene oxazolidinone compound and preparation method thereof

(75) Inventors: Hun Yeong Koh, Seoul (KR); Yong Seo Cho, Seoul (KR); Ae Nim Pae, Seoul (KR); Joo Hwan Cha, Seoul (KR); Hye Yeon Kim, Seoul (KR); Jae Seok Lee, Gyeonggi-Do (KR); Hak Soo Kim, Seoul (KR); Sanghee Kim, Seoul (KR)

Correspondence Address: MORRISON & FOERSTER LLP, 1650 TYSONS BOULEVARD, SUITE 300, MCLEAN, VA, 22102, US

(73) Assignee: Korea Institute of Science and Technology, Seoul (KR)

(21) Appl. No.: 10/694,843

(22) Filed: Oct. 29, 2003

(30) Foreign Application Priority Data
Oct. 29, 2002 (KR) 66268/2002

(51) Int. Cl. A61K 31/433; A61K 31/427; A61K 31/422; C07D 417/14; C07D 413/14

(52) U.S. Cl. 514/362; 514/372; 514/376; 548/128; 548/204; 548/229

(57) ABSTRACT

A methylidene oxazolidinone compound represented by formula (1) or a pharmaceutically acceptable salt thereof, and a preparation method thereof, showing superior antimicrobial activities against gram-positive germs including resistant strains such as methicillic-resistant staphylococcus aureus and vancomycin-resistant enterococcus:

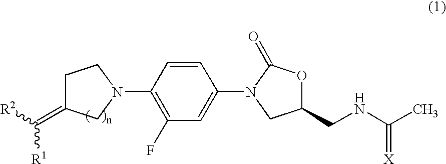

### BACKGROUND OF THE INVENTION

1. Field of the Invention

The present invention relates to a methylidene piperidinyl or pyrrolidinyl oxazolidinone compound or a salt thereof, having anti-microbial activity against gram-positive germs including resistant strains such as methicillin-resistant Staphylococcus aureus (MRSA), vancomycin-resistant Enterococcus faecium (VRE) and the like, and to a preparation method thereof.

2. Description of the Background Art

Researches into antibiotics have been diversely proceeded since World War II. Antibiotics can be divided into β-lactam, aminoglycoside, macrolide, quinolone, tetracycline, glyco-peptide and the like. However, presently used antibiotics have been rapidly losing their activities due to the generation of resistant strains. It is because that high-grade antibiotics have been used in order to treat various infectious diseases in hospitals as people have been easily infected by bacteria, and therefore, such misuses of the antibiotics have made various resistant strains increased rapidly.

Strains such as methicillin-resistant staphylococcus aureus (MRSA), methicillin-resistant Staphylococcus epidermis (MRSE), Enterococcus pneuminiae, quinolone-resistant Staphylococcus aureus (QRSA), vancomycin-resistant Enterococcus (VRE), multi-drug resistant mycobacterium tuberculosis and the like exhibit resistance globally against the most antibiotics which are presently used. Therefore, there is desperate need for a new antibiotic having a new structure and mechanism which are able to solve the above resistance problem.

In 1987, Dupont Co. reported first that Dup-721, a compound of oxazolidinone derivative, showed activities against MRSA and β-lactamase, and compounds included in such group showed anti-microbial activities. However, development for the Dup-721 as an antibiotic was stopped during clinical trial phase 1 due to its toxicity.

Since then, researches into structures and activities of the oxazolidinone compound has been performed by Pharmacia Upjohn, Merck, Bayer and the like. Linezolid (“LZD”), which is a new antibiotic having a new frame, was developed by Upjohn in April, 2000, and has been sold under brand name “Zybox”, which is a new type of antibiotic appeared first since last 35 years. Although the compound shows high pharmaco-kinetic profile, it does not good activity against the resistant strains such as MRSA or VRE.

Therefore, it is required to develop a new compound exhibiting higher activities against wide range of strains which show resistance to conventional antibiotics.

### SUMMARY OF THE INVENTION

Therefore, an object of the present invention is to provide a novel methylidene piperidinyl or pyrrolidinyl oxazolidinone compound or a salt thereof, which can be used as an antibiotic exhibiting higher activities against multi-drug resistant strains, and a preparation method thereof.

Another object of the present invention is to provide a novel oxazolidinone compound or a salt thereof, which has higher anti-microbial activities against methicillin-resistant Staphylococcus aureus (MRSA), methicillin-resistant Staphylococcus epidermidis (MRSE), Enterococcus pneuminiae, quinolone-resistant Staphylococcus aureus (QRSA), vancomycin-resistant Enterococcus faecium (VRE) and the like, and a preparation method thereof.

### DETAILED DESCRIPTION OF THE INVENTION

To achieve the above and other advantages and in accordance with the purpose of the present invention, as embodied and broadly described, there is provided a methylidene piperidinyl or pyrrolidinyl oxazolidinone compound represented by the following formula (1) or a salt thereof, and a preparation method thereof:

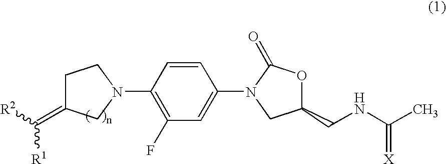

wherein X represents an oxygen or sulfur atom;

R¹ and R² independently represent hydrogen atom, cyano group, alkyl group, halogen atom, acetoxy group, ethoxycarbonyl group, hydroxy group, hydroxyimino group, methoxyimino group, aminoethyl group or a heterocyclic substituent; and

n represents an integer 1 or 2.

In the compound of the above formula (1), the alkyl group may be methyl, ethyl, propyl group or the like, the halogen atom may be chlorine or bromine atom, and the acetoxy group may be substituted with one or more chlorine atoms. In addition, the heterocyclic substituent is a unsaturated 5-membered heterocyclic group containing one or more hetero atoms selected from the group consisting of oxygen, nitrogen and sulfur, and examples of the heterocyclic substituent may include isoxazole, thiophene, thiazole, isothiazole, thiadiazole and the like.

The present invention also includes a pharmaceutically acceptable salt of the compound of formula (1), and the salt may include a salt of methanesulfonate, fumarate, hydrobromide, citrate, maleate, phosphate, sulfate, hydrochloride or sodium.

It was discovered that the compound of the above formula (1) or a salt thereof according to the present invention shows superior activity as twice as the LZD of Upjohn Co. against the MRSA and other Gram-positive germs (See Tables 2 and 3).

Hereinafter, a preparation method of the compound represented by the formula (1) will be described.

The compound of formula (1) can be prepared by reacting an oxazolidinone. intermediate (disclosed in WO. 95/25106) represented by the following formula (2) with a compound represented by the following formula (3) or (4), so as to introduce a methylidene piperidinyl or pyrrolydinyl group thereto:

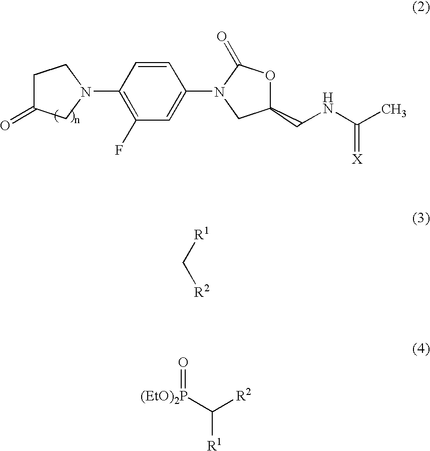

wherein X, R¹, R² and n are the same as defined in the compound of formula (1).

The reaction introducing a methylindene piperidinyl group to the compound of formula (2) with the compound of formula (3) can be represented by the following reaction scheme (I), which uses a reaction known as Knoevenagel condensation.

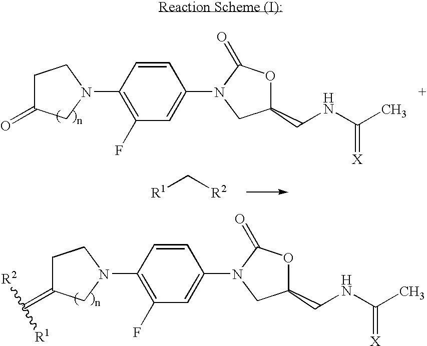

wherein X, R¹, R² and n are the same as defined in the compound of formula (1). It is preferred that R¹ is cyano group, and R² is cyano or ethoxycarbonyl group.

The reaction represented by the reaction scheme (I) can be carried out using dichloromethane or benzene as a solvent, or without using a solvent. As a catalyst of the above reaction, alumina, ammonia, alkyl amine such as triethylamine, aromatic amine such as pyridine, piperidine, potassium fluoride, cerium fluoride, titanium fluoride and the like can be used. It is preferred that the reaction is carried out at room temperature, or at 50-100° C.

In the case that a methylidene piperidinyl or pyrrolidinyl group is introduced to the compound of formula (2) with the compound of formula (4), the reaction can be represented by the following reaction scheme (2), which uses a reaction known as Wadsward-Horner-Emmons' reaction.

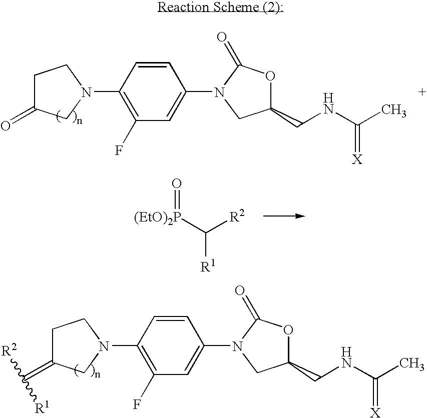

wherein X, R¹, R² and n are the same as defined in the compound of formula (1).

In the reaction represented by the reaction scheme (2), a process for activating phosphonate of formula (4) is required. In the process for activating the phosphonate of formula (4), sodium hydride or potassium t-butoxide can be used as a base, cleanly purified tetrahydrofuran, dimethylethane or dimethylformamide is preferably used as a solvent, and the temperature is preferably maintained at 0° C. or room temperature. After activating the phosphonate of the formula (4), the compound of formula (2) is added thereto, and the resulting mixture is stirred. The reaction can be carried out at room temperature or by refluxing at 40-100° C. range. These all procedures are preferably performed under a nitrogen atmosphere.

The following table 1 shows substituents included in the representative oxazolidinone compounds of the present invention which can be prepared by the reactions represented by the above reaction schemes 1 or 2:

TABLE 1

| Compd. | | |
|---|---|---|
| Nos. | R¹ | R² |
| 1^{a} | CN | CN |
| 2^{a} | CN | CO₂Et |
| 3^{a} | H | CN |
| 4^{a} | CH₃ | CN |
| 5 | CN | (CH₂CO₂Et)^{b} |
| 6 | CN | CN |
| 7 | CN | CO₂Et |
| 8 | H | CN |
| 9 | H | 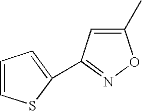 |
| 10 | H | 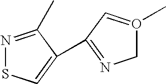 |
| 11 | H | CO₂Et |
| 12 | H | COCH₃ |
| 13 | CH₃ | CO₂Et |
| 14 | H | CO₂Na |
| 15 | Cl | CO₂Et |
| 16 | CN | CH₃ |
| 17 | H | CHO |
| 18 | H | CH(NOH) |
| 19 | H | CH(NOCH₃) |
| 20 | H | C(NOH)CH₃ |
| 21 | H | C(NOCH₃)CH₃ |
| 22 | H | CH(OH)CH₃ |
| 23 | H | CH(OAc)CH₃ |
| 24 | H | CH(OCOCH₂Cl)CH₃ |
| 25 | H | CH(OCOCHCl₂)CH₃ |
| 26^{b} | H | CN |

^{a}n = 1
^{b}X = S

Nomenclatures of the compounds 1-26 shown in the above table 1, are representative compounds of the present invention, are as follows:

Compound 1: N-[[(5S)-3-[3-fluoro-4-(3-dicyanomethylidenepyrrolidin-1-yl)phenyl]-2-oxo-5-oxazolidinyl]methyl]acetamide.

Compound 2: N-[[(5S)-3-[3-fluoro-4-((3-(1-ethoxycarbonyl-1-cyano)-methylidene)pyrrolidin-1-yl)phenyl]-2-oxo-5-oxazolidinyl]methyl]acetamide.

Compound 3: N-[[(5S)-3-[3-fluoro-4-(3-cyanomethylidenepyrrolidin-1-yl)-phenyl]-2-oxo-5-oxazolidinyl]methyl]acetamide.

Compound 4: N-[[(5S)-3-[3-fluoro-4-((3-(1-methyl-1-cyano)methylidene)-pyrrolidin-1-yl)phenyl]-2-oxo-5-oxazolidinyl]methyl]acetamide

Compound 5: N-[[(5S)-3-[3-fluoro-4-(4-(1-cyano-2-ethoxycarbonyl-ethylidene)piperidin-1-yl)phenyl]-2-oxo-5-oxazolidinyl]methyl]acetamide

Compound 6: N-[(5S)-3-[3-fluoro-4-(4-dicyanomethylidenepyrrolidin-1-yl)-phenyl]-2-oxo-5-oxazolidinyl]methylacetamide

Compound 7: N-[[(5S)-3-[3-fluoro-4-((4-(1-ethoxycarbonyl-1-cyano)-methylidene)piperidinyl)phenyl]-2-oxo-5-oxazolidinyl]methyl]acetamide,

Compound 8: N-[[(5S)-3-[3-fluoro-4-(4-cyanomethylidenepiperidinyl)-phenyl]-2-oxo-5-oxazolidinyl]methyl]acetamide,

Compound 9: N-[[(5S)-3-[3-fluoro-4-((4-(3-thiophen-2-yl-5-isoxazolyl)-methylidene)piperidinyl)phenyl]-2-oxo-5-oxazolidinyl]methyl]acetamide,

Compound 10: N-[[(5S)-3-[3-fluoro-4-((4-(3-(3-methyl-isothiazol-4-yl)-iso-xazolyl)methylidene)piperidinyl)phenyl]-2-oxo-5-oxazolidinyl]methyl]acetamide,

Compound 11: N-[[(5S)-3-[3-fluoro-4-(4-ethoxycarbonylmethylidene-piperidinyl)phenyl]-2-oxo-5-oxazolidinyl]methyl]acetamide,

Compound 12: N-[[(5S)-3-[3-fluoro-4-(4-methylcarbonylmethylidene-piperidinyl)phenyl]-2-oxo-5-oxazolidinyl]methyl]acetamide,

Compound 13: N-[[(5S)-3-[3-fluoro-4-(4-(1-ethoxycarbonylethylidene)-piperidinyl)phenyl]-2-oxo-5-oxazolidinyl]methyl]acetamide,

Compound 14: N-[[(5S)-3-[3-fluoro-4-(4-carboxymethylidenepiperidinyl)-phenyl]-2-oxo-5-oxazolidinyl]methyl]acetamide,

Compound 15: N-[[(5S)-3-[3-fluoro-4-((4-(1-ethoxycarbonyl-1-chloro)-methylidene)piperidinyl)phenyl]-2-oxo-5-oxazolidinyl]methyl]acetamide,

Compound 16: N-[[(5S)-3-[3-fluoro-4-(4-(1-cyanoethylidene)piperidinyl)-phenyl]-2-oxo-5-oxazolidinyl]methyl]acetamide,

Compound 17: N-[[(5S)-3-[3-fluoro-4-(4-(2-oxoethylidene)piperidinyl)-phenyl]-2-oxo-5-oxazolidinyl]methyl]acetamide,

Compound 18: N-[[(5S)-3-[3-fluoro-4-(4-(2-hydroxyiminoethylidene)-piperidinyl)phenyl]-2-oxo-5-oxazolidinyl]methyl]acetamide,

Compound 19: N-[[(5S)-3-[3-fluoro-4-(4-(2-methoxyiminoethylidene)-piperidinyl)phenyl]-2-oxo-5-oxazolidinyl]methyl]acetamide,

Compound 20: N-[[(5S)-3-[3-fluoro-4-(4-(2-hydroxyiminopropylidene)-piperidinyl)phenyl]-2-oxo-5-oxazolidinyl]methyl]acetamide,

Compound 21: N-[[(5S)-3-[3-fluoro-4-(4-(2-methoxyiminopropylidene)-piperidinyl)phenyl]-2-oxo-5-oxazolidinyl]methyl]acetamide,

Compound 22: N-[[(5S)-3-[3-fluoro-4-(4-(2-hydroxypropylidene)-piperidinyl)-phenyl]-2-oxo-5-oxazolidinyl]methyl]acetamide,

Compound 23: N-[[(5S)-3-[3-fluoro-4-(4-(2-acetoxypropylidene)-piperidinyl)phenyl]-2-oxo-5-oxazolidinyl]methyl]acetamide,

Compound 24: N-[[(5S)-3-[3-fluoro-4-(4-(2-(chloroacetoxy)propylidene)-piperidinyl)-phenyl]-2-oxo-5-oxazolidinyl]methyl]acetamide,

Compound 25: N-[[(5S)-3-[3-fluoro-4-(4-(2-(dichloroacetoxy)propylidene)-piperidinyl)-phenyl]-2-oxo-5-oxazolidinyl]methyl]acetamide,

Compound 26: N-[[(5S)-3-[3-fluoro-4-(4-(cyanomethylidene)piperidinyl)-phenyl]-2-oxo-5-oxazolidinyl]methyl]thioacetamide.

The compound of formula (1) according to the present invention also includes salts of the above compounds 1-26.

### EXAMPLES

Hereinafter, the present invention will be described in more detail by the following Examples. However, the Examples are to illustrate the present invention, and not to limit the scope of the present invention thereto.

### Example 1

Preparation of N-[[(5S)-3-[3-fluoro-4-(3-dicyanomethylidenepyrrolidin-1-yl)phenyl]-2-oxo-5-oxazolidinyl]methyl]acetamide and hydrochloride salt thereof

N-{3-[3-fluoro-4-(3-oxo-pyrrolidin-1-yl)-phenyl]-2-oxo-oxazolidin-5-yl-methyl}acetamide (50 mg, 0.15 mmol), Al₂O₃ (200 mg, Basic, I™, Aldrich), malononitrile (5 g, excess) were mixed, and the resulting mixture was then stirred for 30 minutes at 40° C. The reaction mixture was washed with water and extracted with dichloromethane. Organic extract was dried with anhydrous magnesium sulfate and then concentrated. The concentrated residue was purified by column chromatography (silica, EtOAc:MeOH=40:1), to obtain 29.4 mg of desired product (51% yield). NMR data identifying the product were as follows:

¹H NMR (CDCl₃): δ 7.48(d, J=14.7, 1H), 7.11(d, J=1.0.5, 1H), 6.78(t, J=3.0, 1H), 5.97(s, 1H), 4.78(m, 1H), 4.43(s, 2H), 4.03(t, J=9, 1H), 3.74(m, 3H), 3.61(m, 2H), 3.20(t, 2H), 2.00(s, 3H).

Hydrochloride salt was obtained by treating the product with ethyl ether saturated with hydrogen chloride gas.

### Example 2

Preparation of N-[[(5S)-3-[3-fluoro-4-(3-(1-cyano-1-ethoxycarbonyl) methyl-idenepyrrolidin-1-yl)phenyl]-2-oxo-5-oxazolidinyl]methyl]acetamide and hydro-chloride salt thereof

N-{3-[3-fluoro-4-(3-oxo-pyrrolidin-1-yl)-phenyl]-2-oxo-oxazolidin-5-yl-methyl}acetamide (50 mg, 0.15 mmol), ethyl cyanoacetate (5 ml, excess) and Al₂O₃ (200 mg, Basic, I™, Aldrich) were mixed, and the resulting solution was stirred for 13 hours at room temperature. The reaction mixture was washed with water and then extracted with dichloromethane. Organic extract was dried with anhydrous magnesium sulfate and concentrated. The concentrated residue was purified by column chromatography (silica, EtOAc:MeOH=40:1), to obtain 32.3 mg of desired product (50% yield). NMR data identifying the product were as follows:

¹H NMR (CDCl₃, 300 MHz): δ 7.45(d, J=15.12, 1H), 7.09(d, J=8.61, 1H), 6.80(m, 1H), 6.00(s, 1H), 4.77(m, 1H), 4.60(s, 2H), 4.31(m, 2H), 4.02(t, J=8,80, 1H), 3.75(m, 2H), 3.61(m, 3H), 3.18(m, 2H), 2.03 (s, 3H), 1.37(t, J=7.14, 3H)

Hydrochloride salt was obtained by treating the product with ethyl ether saturated with hydrogen chloride gas.

### Example 3

Preparation of N-[[(5S)-3-[3-fluoro-4-(3-cyanomethylidenepyrrolidin-1-yl)-phenyl]-2-oxo-5-oxazolidinyl]methyl]acetamide and hydrochloride salt thereof

Potassium t-butoxide (33:46 mg, 0.30 mmol) was dissolved in 3 ml of THF in a reactor under a nitrogen atmosphere, and the resulting solution was cooled down to −78° C. While the temperature of the reactor was maintained at −78° C., a solution of diethyl cyanomethyl phosphonate (52.83 mg, 0.33 mmol) in 3 ml of THF was slowly added thereto, and the resulting solution was stirred for one hour. A solution of N-{3-[3-fluoro-4-(3-oxo-pyrrolidin-1-yl)phenyl]-2-oxo-oxazolid in-5-ylmethyl}acetamide (500 mg, 1.49 mmol) in 9 ml of THF was added to the reactor for 20 minutes, and the resulting solution was stirred for 3 hours. The reaction was ended by adding water, and the reaction mixture was extracted with ethyl acetate. Organic extract was dried with magnesium sulfate and concentrated under a reduced pressure. The concentrated residue was purified by column chromatography (EtOAc:MeOH=10:1) using silica gel (230-400 mesh) neutralized with triethylamine, to obtain 50 mg of desired product (59% yield). NMR data identifying the product were as follows:

¹H NMR (CDCl₃, 300 MHz): 6 7.40(dd, J=15.12 1H), 7.05(d, J=8.76, 1H), 6.70(m, 2H), 5.38(d, J=2.37, 1H), 4.77(m, 1H), 4.23(s, 1H), 4.13(s, 1H), 4.01(t, J=8.91, 1H), 3.76(t, J=7.88, 1H), 3.66(d, J=2.66, 2H), 3.48(m, 2H), 2.92(m, 2H) 2.02 (s, 3H);

¹³C NMR (CDCl₃, 300 MHz): δ 171.69, 166.61, 154.95, 154.84, 151.40, 133.31, 13.20, 133.17, 131.38, 131.25, 117.10, 117.07, 117.03, 116.37, 116.40, 114.80, 114.76, 108.41, 108.06, 91.91, 91.58, 77.89, 77.46, 77.05, 72.40, 56.02, 55.93, 50.04, 49.98, 49.66, 49.60, 48.13, 42.31, 32.96, 32.12, 23.40.

Hydrochloride salt was obtained by treating the product with ethyl ether saturated with hydrogen chloride gas.

### Example 4

Preparation of N-(3-{4-[3-(cyano-methyl-methylene)-pyrrolid in-1-yl]-3-fluorophenyl}-2-oxo-oxazolidin-5-ylmethyl)acetamide and hydrochloride salt thereof

Under a nitrogen atmosphere, potassium t-butoxide (133.8 mg, 0.24 mmol) was dissolved in 5 ml of THF, and a solution of diethyl cyanoethyl phosphonate (214.4 mg, 1.22 mmol) in 5 ml of THF was slowly added thereto. The resulting solution was stirred for 30 minutes and then cooled down to −78° C. A solution of N-{3-[3-fluoro-4-(3-oxo-pyrrolid in-1-yl)phenyl]-2-oxo-oxazolidin-5-yl-methyl}acetamide (80.0 mg, 0.24 mmol) in 16 ml of THF was added thereto for 20 minutes, and the resulting solution was stirred for 8 hours. The reaction was ended by adding water, and the reaction mixture was extracted with ethyl acetate. Organic extract was dried with magnesium sulfate and concentrated under a reduced pressure. The concentrated residue was purified by column chromatography (EtOAc:MeOH=40:1) using silica gel (230-400 mesh), to obtain 88.0 mg of product (98% yield). NMR data identifying the product were as follows:

¹H NMR (CDCl₃, 300 MHz): δ 7.45(d, J=15.12 1H), 7.09(d, J=8.61, 1H), 6.80(m, 1H), 6.00(s, 1H), 4.77(m, 1H), 4.60(s, 2H), 4.31(m, 2H), 4.02(t, J=8.80, 1H), 3.75(m, 2H), 3.61(m, 3H), 3.18(m, 2H), 2.03(s, 3H), 1.37 (t, J=7.14, 3H).

Hydrochloride salt was obtained by treating the product with ethyl ether saturated with hydrogen chloride gas.

### Example 5

Preparation of N-[[(5S)-3-[3-fluoro-4-(4-(1-cyano-2-ethoxycarbonyl-ethylidene)piperid in-1-yl)phenyl]-2-oxo-5-oxazolidinyl]methyl]acetamide

120 mg of 60% NaH (3.01 mmol) was dissolved in 6 ml of purified tetrahydrofuran, and a solution of triethyl 3-cyano-3-(diethoxyphosphoryl)-propionic acid ethyl ester (791 mg, 3.01 mmol) in 0.5 ml of tetrahydrofuran was slowly added thereto. The resulting solution was then stirred for two and a half hours at room temperature. N-[(5S)-3-[3-fluoro-4-(4-oxopiperidinyl)phenyl]-2-oxo-5-oxazolidinyl]methylacetamide (300 mg, 0.86 mmol) was added to the above solution, and the resulting solution was stirred for 20 hours at room temperature. After adding water to the reaction mixture, the water layer was extracted with dichloromethane. Organic extract was dried with magnesium sulfate and concentrated under a reduced pressure. The concentrated residue was purified by column chromatography using 10% methanol in ethyl acetate, to obtain 84.8 mg (22% yield)of the desired product as a solid. NMR data identifying the product were as follows:

¹H NMR (300 MHz, CDCl₃): δ 7.43 (dd, J=14.2 Hz, 2.6 Hz, 1H), 7.05 (dd, J=8.8 Hz, 1.7Hz, 1H), 6.91 (t, J=9.1 Hz, 1H), 6.40 (t, J=6.3 Hz, 1H), 4.77 (m, 1H), 4.00 (m, 1H), 3.76 (m, 1H), 3.64 (m, 2H), 3.12 (m, 4H), 2.77 (t, J=5.4 Hz, 2H), 2.54 (t, J=5.5 Hz, 2H), 2.00 (s, 3H), 1.93 (s, 3H).

Preparation of N-[(5S)-3-[3-fluoro-4-(4-(1-cyano-4-hydroxybutylidene)-piperidinyl)phenyl]-2-oxo-5-oxazolidinyl]methylacetamide

N-[(5S)-3-[3-fluoro-4-(4-(1-cyano-2-ethoxycarbonylethyl-idene)piperidin-yl)phenyl]-2-oxo-5-oxazolidinyl]methylacetamide (48 mg, 0.11 mmol) was dissolved in 3 ml of tetrahydrofuran/water (1/2), and sodium borohydride (10 mg, 0.27 mmol) was added thereto. The reaction mixture was stirred for 3 hours at 0° C. and then stirred for 16 hours at room temperature. Saturated aqueous ammonium chloride solution was added to the reaction mixture. The solution was stirred for 5 minutes, and then extracted with ethyl acetate. Organic extract was dried with anhydrous magnesium sulfate and then concentrated under a reduced pressure, to obtain 38.9 mg of yellow solid (89% yield).

Preparation of N-[(5S)-3-[3-fluoro-4-(4-(1-cyano-4-methanesulfonyloxy) butylidene)piperidinyl)phenyl]-2-oxo-5-oxazolidinyl]methylacetamide

N-[(5S)-3-[3-fluoro-4-(4-(1-cyano-4-hydroxy-butylidene)piperidinyl)-phenyl)-2-oxo-5-oxazolidinyl]methylacetamide (38.9 mg, 0.09 mmol) and triethylamine (48 μl, 0.35 mmol) were dissolved in 1 ml of dichloromethane, and 21 μl (0.27 mmol) of methanesulfonyl chloride was slowly added thereto at 0° C. The resulting solution was stirred for 2 hours and then washed with water. Water layer was extracted with dichloromethane. Organic extract was dried with anhydrous magnesium sulfate and then concentrated under a reduced pressure, to obtain 46 mg (99% yield) of desired product.

Preparation of N-[(5S)-3-[3-fluoro-4-(4-(1-cyano-4-azido)butylidene)-piperidinyl)phenyl]-2-oxo-5-oxazolidinyl]methylacetamide 46 mg (0.09 mmol) of N-[(5S)-3-[3-fluoro-4-(4-(1-cyano-2-methansulfonyloxy)butylidene)piperidinyl]phenyl)-2-oxo-5-oxazolidinyl]methylacet-amide was dissolved in 1 ml of N,N-dimethyl formamide, and 48 mg (0.74 mmol) of sodium azide was added thereto. The resulting solution was stirred for 18 hours at 80° C. Water was poured onto the reaction mixture, and water layer was then extracted with ethyl acetate. Organic extract was dried with anhydrous magnesium sulfate and then concentrated under a reduced pressure, to obtain 33.3 mg of desired product (81% yield).

Preparation of N-[(5S)-3-[3-fluoro-4-(4-(1-cyano-4-aminopropylidene) piperidinyl)phenyl]-2-oxo-5-oxazolidinyl]methylthioacetamide and hydrochloride salt thereof.

33.3 mg (0.08 mmol) of N-[(5S)-3-[3-fluoro-4-(4-(1-cyano-4-azido)-butylidene)piperidinyl]phenyl)-2-oxo-5-oxazolidinyl]methylacetamide was dissolved in 1 ml of tetrahydrofuran/water (1/3), and 35 mg (0.30 mmol) of indium and 290 μl of 6N hydrochloric acid were added thereto. The resulting solution was stirred for 10 hours at room temperature. The reaction mixture was filtered under a reduced pressure. The filtrate was washed with ethyl acetate several times and then neutralized with 3N sodium hydroxide solution. The water layer was then extracted with ethyl acetate. Organic extract was dried with anhydrous magnesium sulfate and then concentrated under a reduced pressure, to obtain 13.0 mg of desired product (41% yield). NMR data identifying the product as follows:

¹H NMR (300 MHz, CDCl₃): δ 7.43 (dd, J=14.0, 2.4 Hz, 1H), 7.02 (d, J=8.8 Hz, 1H), 6.89 (t, J=9.2 Hz, 1H), 6.73 (s, br, 1H), 4.74 (m, 1H), 4.98 (t, J=8.9 Hz, 1H), 3.75 (t, J=9.2 Hz, 1H), 3.62 (t, J=5.5 Hz, 2H), 3.49 (t, J=6.6 Hz, 2H), 3.11 (m, 4H), 2.79 (t, J=5.9 Hz, 2H), 2.63-2.51 (m, 4H), 1.96 (s, 3H).

Hydrochloride salt was obtained by treating the product with ethyl ether saturated with hydrogen chloride gas.

### Example 6

Preparation of N-[[(5S)-3-[3-fluoro-4-(4-dicyanomethylidenepiperidinyl) phenyl]-2-oxo-5-oxazolidinyl]methyl]acetamide and hydrochloride salt thereof N-[(5S)-3-[3-fluoro-4-(4-oxopiperidinyl)phenyl]-2-oxo-5-oxazolidinyl]methylacetamide (20.0 mg, 0.06 mmol) was dissolved in 1 ml of dichloromethane, and malononitrile (3.8 mg, 0.06 mmol) and A1₂0₃ (17.2 mg, Basic, I™, Aldrich) were added thereto. The resulting solution was stirred for 18 hours at 40° C., and then washed with water. The water layer was extracted with dichloromethane, and organic extract was dried with anhydrous magnesium sulfate, which was then filtered off. The filtrate was concentrated under a reduced pressure, to obtain 23.7 mg (99% yield) of desired product. NMR and IR data identifying the product were as follows:

¹H NMR (300 MHz, CDCl₃): δ 7.47 (dd, J=14.0 Hz, 1.2 Hz, 1H), 7.09 (dd, J=8.7, 1.1 Hz, 1H), 6.92 (t, J=9.1 Hz, 1H), 6.31 (s, br, 1H), 4.77 (m, 1H), 3.99 (t, J=9.1 Hz, 1H), 3.76 (t, J=7.1Hz, 1H), 3.67 (m, 2H), 3.26 (t, J=5.5 Hz, 4H), 2.92 (t, J=5.4 Hz, 4H), 1.99 (s, 3H);

IR (KBr, cm⁻¹): 3300, 2924, 2232, 1750, 1656, 1418, 1382, 1216, 866, 752.

Hydrochloride salt was obtained by treating the product with ethyl ether saturated with hydrogen chloride gas.

### Example 7

Preparation of N-[[(5S)-3-[3-fluoro-4-((4-(1-ethoxycarbonyl-cyano)methylidene)piperidinyl)phenyl]-2-oxo-5-oxazolidinyl]methyl]acetamide and hydrochloride salt thereof

N-[(5S)-3-[3-fluoro-4-(4-oxopiperid inyl)phenyl]2-oxo-5-oxazol id inyl]methyl-acetamide (2.42g, 6.93 mmol), ethyl cyanoacetate (6 ml, excess) and Al₂O₃ (2.08 g, 20.4 mmol, Basic, I™, Aldrich) were put into a reactor, and the resulting solution was then stirred for 24 hours at 90-100° C. The reaction mixture is filtered using cellite. The filtrate was washed with water and then extracted with dichloromethane. Organic extract was dried with anhydrous magnesium sulfate and then concentrated under a reduced pressure. The concentrated residue was purified by column chromatography using 10% methanol-ethyl acetate, to obtain 1.89 g (61% yield) of desired product. NMR and IR data identifying.the product were as follows:

¹H NMR (CDCl₃,300 MHz,): δ 7.42 (dd, J=14.1 Hz, J=2.6 Hz, 1H), 7.04 (dd, J=8.8 Hz, J=2.1 Hz, 1H), 6.89 (t, J=9.1 Hz, 1H), 6.68 (t, J=5.3 Hz, 1H), 4.76 (m, 1H), 4.27 (q, J=7.1 Hz, 2H), 4.00 (t, J=9.OHz, 1H), 3.74 (m, 1H), 3.62 (m, 2H), 3.30-3.22 (m, 4H), 3.16 (t, J=5.5 Hz, 2H), 2.91 (t, J=5.7 Hz, 2H), 2.00 (s, 3H), 1.32 (t, J=7.1 Hz, 3H);

IR (KBr, cm⁻¹): 924, 2232, 1750, 1656, 1518, 1418, 1382, 1216, 866, 752.

Hydrochloride salt was obtained by treating the product with ethyl ether saturated with hydrogen chloride gas.

### Example 8

Preparation of N-[[(5S)-3-[3-fluoro-4-(4-cyanomethylidenepiperidinyl)-phenyl]-2-oxo-5-oxazolidinyl]methyl]acetamide salt thereof

80% NaH (12.9 mg, 0.43 mmol) was dissolved in 0.5 ml of purified tetrahydrofuran, and diethyl cyanomethyl phosphonate (55.7 mg, 0.32 mmol) was slowly added thereto. The resulting solution was stirred for one hour at room temperature. N-[(5S)-3-[3-fluoro-4-(4-oxopiperidinyl)phenyl]-2-oxo-5-oxazolidinyl]-methylacetamide (100 mg, 0.29 mmol) was added to the above solution, Which was then stirred for 3 hours at room temperature. Water was poured onto the reaction mixture, and water layer was then extracted with dichloromethane. Organic extract was dried with anhydrous magnesium sulfate, which was then filtered off, and the filtrate was concentrated under a reduced pressure. The concentrated residue was purified by column chromatography using 10% methanol-ethyl acetate, to obtain 105 mg (64% yield) of desired product. NMR, IR and Mass data identifying the product were as follows:

¹H NMR (300 MHz, CDCl₃): δ 7.47 (dd, J=14.1 Hz, J=2.55 Hz, 1H), 7.16 (dd, J=8.79, J=1.62 Hz, 1H), 6.94 (t, J=2.49 Hz, 1H), 6.23 (t, J=6.09 Hz, 1H) (s, 1H), 4.78 (m, 1H), 4.16-4.00 (m, 1H), 3.79-3.72 (m, 1H), 3.69-3.58 (m, 2H), 3.20-3.10 (m, 4H), 2.78 (t, J=5.28 Hz, 2H), 2.54 (t, J=5.28 Hz, 2H), 2.00 (s, 3H);

¹³C NMR (300 MHz, CDCl₃): δ 171.91 (—NHCOCH₃), 164.45 (Ph, C-F), 157.01 (isoxazole carbonyl), 155.09 (piperidinyl C=), 114.65 (CN), 108.14 (H(CN)C═), 23.07 (—NHCOCH₃);

IR (KBr, cm⁻¹) 2232 (CN);

HRMS (FAB⁺) C₁₉H₂₂FN₄O₃ calculated: 373.1598, found: 373.1676.

Hydrochloride salt was obtained by treating the product with ethyl ether saturated with hydrogen chloride gas.

### Example 9

Preparation of N-[[(5S)-3-[3-fluoro-4-((4-(3-thiophen-2-yl-5-isoxazolyl)-methylidene)piperidinyl)phenyl]-2-oxo-5-oxazolidinyl]methyl]acetamide and hydro-chloride salt thereof

80% NaH (17.2 mg, 0.57 mmol) was dissolved in 1.0 ml of purified tetrahydrofuran, and diethyl 3-(2-thiophenyl)-5-isoxazolmethylene phosphonate (129 mg, 0.43 mmol) was slowly.added thereto. The resulting solution was stirred for one hour at room temperature. N-[(5S)-3-[3-fluoro-4-(4-oxopiperidinyl)phenyl]-2-oxo-5-oxazolidinyl]methylacetamide (100 mg, 0.29 mmol) was added to the above solution, which was then stirred for 20 hours at room temperature. Water was poured onto the reaction mixture, and water layer was extracted with dicholoromethane. Organic extract was dried with anhydrous magnesium sulfate, which was then filtered off, and the filtrate was concentrated under a reduced pressure. The concentrated residue was purified by column chromatography using 10% methanol-ethyl acetate, to obtain 42.4 mg (20% yield) of desired product. NMR data identifying the product were as follows:

¹H NMR (300 MHz, CDCl₃): δ 7.43 (dd, J=17.0, 13.5 Hz, 2H), 7.11 (t, J=3.5 Hz, 1H), 7.04 (d, J=9.0 Hz, 1H), 6.92 (t, J=8.8 Hz, 1H), 6.32 (t, J=7.2 Hz, 1H), (s, 1H), 4.77 (m, 1H), 4.01 (m, 1H), 3.75 (m, 1H), 3.63 (m, 2H), 3.15 (m, 4H), 2.95 (t, J=4.5 Hz, 2H), 2.52 (t, J=4.5 Hz, 2H), 2.01 (s, 1H).

Hydrochloride salt was obtained by treating the product with ethyl ether saturated with hydrogen chloride gas.

### Example 10

Preparation of N-[[(5S)-3-[3-fluoro-4-((4-(3-(3-methyl-isothiazol-4-yl)-isoxazolyl)methylidene)piperidinyl)phenyl]-2-oxo-5-oxazolidinyl]methyl]acetamide and hydro-chloride salt thereof

80% NaH (17.2 mg, 0.57 mmol) was dissolved in 1.0 ml of cleanly purified tetrahydrofuran, and 3-(2-isothiophenyl)-5-isoxazolmethylene phosphonate (136 mg, 0.43 mmol) was slowly added thereto. The resulting solution was stirred for one hour at room temperature. N-[(5S)-3-[3-fluoro-4-(4-oxopiperidinyl)phenyl]-2-oxo-5-oxazolidinyl]methylacetamide (100 mg, 0.29 mmol) was added to the above solution, which was then stirred for 20 hours at room temperature. Water was poured onto the reaction mixture, and water layer was extracted with dicholoromethane. Organic extract was dried with magnesium sulfate, which was then filtered off, and the filtrate was concentrated under a reduced pressure. The concentrated residue was purified by column chromatography using 10% methanol-ethyl acetate, to obtain 31.9 mg (15% yield) of desired product. NMR data identifying the product were as follows:

¹H NMR (300 MHz, CDCl₃): δ 8.87 (s, 1H), 7.3 (dd, J=18.0 Hz, J=2.46 Hz, 1H), 7.07 (dd, J=18 Hz, J=1.8 Hz, 1H), 6.95 (s, 1H), 6.38 (t, J=6.1 Hz, 1H), 6.32 (s, 1H), 6.23 (s, 1H), 4.77 (s, 1H), 4.07-3.98 (m, 1H), 3.79-3.71 (m, 1H), 3.69-3.52 (m, 2H), 3.18 (t, J=5.34 Hz, 4H), 2.99 (t, J=5.1 Hz, 2H), 2.60 (t, J=5.1 Hz, 2H), 2.01 (s, 3H).

Hydrochloride salt was obtained by treating the product with ethyl ether saturated with hydrogen chloride gas.

### Example 11

Preparation of N-[[(5S)-3-[3-fluoro-4-(4-ethoxycarbonylmethylidene-piperidinyl)phenyl]-2-oxo-5-oxazolidinyl]methyl]acetamide and hydrochloride salt thereof

80% NaH (3.1 mg, 0.10 mmol) was dissolved in 1.0 ml of cleanly purified dimethoxyethane, and triethyl phosphonoacetate (2.1 ml, 0.10 mmol) was slowly added thereto. The resulting solution was stirred for 2 hours at room temperature. N-[(5S)-3-[3-fluoro-4-(4-oxopiperidinyl)phenyl]-2-oxo-5-oxazolidinyl]methylacet-amide (30.0 mg, 0.09 mmol) was added to the above solution, which was then stirred for 2 hours at room temperature. Water was poured onto the reaction mixture, and water layer was extracted with dicholoromethane. Organic extract was dried with magnesium sulfate, which was then filtered off, and the filtrate was concentrated under a reduced pressure. The concentrated residue was purified by column chromatography using 10% methanol-ethyl acetate, to obtain 23.2 mg (64% yield) of desired product. NMR data identifying the product were as follows:

¹H NMR (300 MHz, CDCl₃): δ 7.42 (dd, J=12.0 Hz, J=3.0 Hz, 1H), 7.05 (dd, J=12.0 Hz, J=3.0 Hz, 1H), 6.92 (t, J=9.0 Hz, 1H), 6.47 (t, J=4.5Hz, 1H), 5.71 (s, 1H), 4.78 (m, 1H), 4.17 (q, J=7.5 Hz, 2H), 3.78-3.60 (m, 5H), 3.11 (s, br, 4H), 2.49 (t, J=3.0 Hz, 2H), 2.01 (s, 3H), 1.39 (t, J=7.5 Hz, 3H).

Hydrochloride salt was obtained by treating the product with ethyl ether saturated with hydrogen chloride gas.

### Example 12

Preparation of N-[[(5S)-3-[3-fluoro-4-(4-methylcarbonylmethylidene-5 piperidinyl)phenyl]-2-oxo-5-oxazolidinyl]methyl]acetamide and hydrochloride salt thereof

80% NaH (6.0 mg, 0.20 mmol) was dissolved in 1.0 ml of cleanly purified tetrahydrofuran, and diethoxy 2-oxopropylphosphonate (38.5 μl, 0.20 mmol) was slowly added thereto. The resulting solution was stirred for 2 and a half hours at room temperature. N-[(5S)-3-[3-fluoro-4-(4-oxopiperidinyl)phenyl]-2-oxo-5-oxazol-idinyl]methylacetamide (57.5 mg, 0.17 mmol) was added to the above solution, which was then stirred for 3 hours at room temperature. Water was poured onto the reaction mixture, and water layer was extracted with dicholoromethane. Organic extract was dried with magnesium sulfate, which was then filtered off, and the filtrate was concentrated under a reduced pressure, to obtain 58.5 mg (91% yield) of desired product of yellow color. NMR data identifying the product were as follows:

¹H NMR (300 MHz, CDCl₃): δ 7.41 (dd, J=14.2 Hz, 2.1 Hz, 1H), 7.03 (dd, J=8.8 Hz, 2.6 Hz, 1H), 6.92 (t, J=9.1 Hz, 1H), 6.42 (t, J=6.0 Hz, 1H), 6.09 (s, 1H), 4.76 (m, 1H), 4.00 (m, 1H), 3.74 (dd, J=6.8 Hz, 2.4 Hz, 1H), 3.65 (m, 2H), 3.170-3.09 (m, overlap, 6H), 2.45 (t, J=5.1 Hz, 2H), 2.20 (s, 3H), 2.01 (s, 3H).

Hydrochloride salt was obtained by treating the product with ethyl ether saturated with hydrogen chloride gas.

### Example 13

Preparation of N-[[(5S)-3-[3-fluoro-4-(4-(1-ethoxycarbonylethylidene)-piperidinyl)phenyl]-2-oxo-5-oxazolid inyl]methyl]acetamide and hydrochloride salt thereof

80% NaH (6.0 mg, 0.20 mmol) was dissolved in 1.0 ml of cleanly purified dimethoxyethane, and triethyl 2-phosphonoacetate (43 μl, 0.20 mmol) was slowly added thereto. The resulting solution was stirred for 2 hours at room temperature. N-[(5S)-3-[3-fluoro-4-(4-oxopiperidinyl)phenyl]-2-oxo-5-oxazolidinyl]methylacet-amide (50.0 mg, 0.14 mmol) was added to the above solution, which was then stirred for 20 hours at room temperature. Water was poured onto the reaction mixture, and water layer was extracted with dicholoromethane. Organic extract was dried with magnesium sulfate, which was then filtered off, and the filtrate was concentrated under a reduced pressure. The concentrated residue was purified by column chromatography using 10% methanol-ethyl acetate, to obtain 9.2 mg (15% yield) of desired product. NMR data identifying the product were as follows:

¹H NMR (300 MHz, CDCl₃): δ 7.43 (d, J=14.1 Hz, 1H), 7.09-6.93 (m, 2H), 6.20 (t, J=2.97 Hz, 1H), 4.77 (m, 1H), 4.21 (q, J=14.3 Hz, 2H), 4.01 (t, J=8.79 Hz, 1H), 3.79-3.60 (m, 3H), 3.10 (s, br, 4H), 2.81 (s, br, 2H), 2.54 (s, br, 2H), 2.54 (s, br, 2H), 2.01 (s, 3H), 1.91 (s, 3H), 1.31 (t, J=14.3 Hz, 3H).

Hydrochloride salt was obtained by treating the product with ethyl ether saturated with hydrogen chloride gas.

### Example 14

Preparation of allyl diethoxyphosphonyl acetate

Diethyl phosphono acetic acid (1.0 g, 5.10 mmol) was dissolved in 5 ml of N,N-dimethylformamide, and potassium carbonate (1.06 g, 7.65 mmol) and allyl bromide (1.0 ml, 11.7 mmol) were added thereto. The resulting solution was stirred for one hour at 30-40° C., and then cooled down to room temperature. Water was poured to the mixture, and water layer was extracted with ethyl acetate. Organic extract was dried with magnesium sulfate, which was then filtered off, and the filtrate was then concentrated under a reduced pressure, to obtain 110 mg (59% yield) of yellow product.

Preparation of N-[[(5S)-3-[3-fluoro-4-(4-carboxymethylidenepiperidinyl)-phenyl]-2-oxo-5-oxazolidinyl]methyl]acetamide and sodium salt thereof

(5S)-N-[3-[fluoro-4-(4-allyloxycarbonylmethylidene)piperidinyl]phenyl]-2-oxo-5-oxazolidinyl]methylacetamide (98 mg, 0.23 mmol), sodium 2-ethyl hexanoate (55.8 mg, 0.34 mmol), triphenyl phosphine (6.0 mg, 0.02 mmol) and tetrakis(triphenyl phosphine) palladium (0) (5.2 mg, 0.005 mmol) were dissolved in 1 ml of dichloromethane. The resulting solution was stirred for 20 hours at room temperature. Acetone was then added to the above solution to form solid, which was filtered and washed with ether, to obtain 55.8 mg (59% yield) of desired product as a white solid. NMR data identifying the product.were as follows:

¹H NMR (300 MHz, CD₃OD): δ 7.47 (dd, J=14.5 MHz, J=1.86 MHz, 1H), 7.12 (dd, J=8.79 MHz, J=1.14 MHz, 1H), 7.03 (t, J=9.09 MHz, 1H), 5.73 (s, 1H), 4.76 (m, 1H), 4.10 (t, J=9.06 MHz, 1H), 3.77 (dd, J=9.06 MHz, J=6.57 MHz, 1H), 3.54 (d, J=4.95 MHz, 2H), 3.06 (m, 4H), 2.95 (d, J=4.74 MHz, 2H), 2.38 (t, J=12.2 MHz, 2H), 1.95 (s, 3H).

### Example 15

Preparation of N-[[(5S)-3-[3-fluoro-4-((4-(1-ethoxycarbonyl-1-chloro)-methylidene)piperidinyl)phenyl]-2-oxo-5-oxazolidinyl]methyl]acetamide and hydrochloride salt thereof

80% NaH (7.2 mg, 0.24 mmol) was dissolved in 1.0 ml of purified tetrahydrofuran, and triethyl 2-chloro-2-phosphonoacetate (51.4 μl, 0.24 mmol) was slowly added thereto. The resulting solution was stirred for 1 and a half hours at room temperature. N-[(5S)-3-[3-fluoro-4-(4-oxopiperidinyl)phenyl]-2-oxo-5-oxazolidinyl]methylacetamide (60.0 mg, 0.17 mmol) was added to the above solution, which was then stirred for 4 hours at room temperature. Water was poured onto the reaction mixture, and water layer was extracted with dicholoromethane. Organic extract was dried with anhydrous magnesium sulfate, which was then filtered off, and the filtrate was concentrated under a reduced pressure. The concentrated residue was purified by column chromatography using 10% methanol-ethyl acetate, to obtain 30 mg (38% yield) of desired product. NMR data identifying the product were as follows:

¹H NMR (300 MHz, CDCl₃): δ 7.41 (d, J=14.2 Hz, 2.2 Hz, 1H), 7.03 (dd, J=8.8 Hz, 1.8Hz, 1H), 6.91 (t, J=9.2 Hz, 1H), 6.57 (t, J=6.0 Hz, 1H), 4.76 (m, 1H), 4.26 (q, J=7.1 Hz, 2H), 3.98 (t, J=6.2 Hz, 1H), 3.74 (t, J=8.8 Hz, 1H); 3.62 (m, 2H), 3.12 (m, 4H), 2.98 (t, J=5.5 Hz, 2H), 2.79 (t, J=5.5 Hz, 2H), 1.98 (s, 3H), 1.33 (t, J=7.1 Hz, 3H).

Hydrochloride salt was obtained by treating the product with ethyl ether saturated with hydrogen chloride gas.

### Example 16

Preparation of N-[[(5S)-3-[3-fluoro-4-(4-(1-cyanoethylidene)piperidinyl)-phenyl]-2-oxo-5-oxazolidinyl]methyl]acetamide and hydrochloride salt thereof

80% NaH (7.4 mg, 0.246 mmol) was dissolved in 1.0 ml of cleanly purified tetrahydrofuran, and diethyl 2-cyanomethylphosphonoacetate (37 μl, 0.21 mmol) was slowly added thereto. The resulting solution was stirred for one and a half hours at room temperature. N-[(5S)-3-[3-fluoro-4-(4-oxopiperidinyl)phenyl]-2-oxo-5-oxazolidinyl]methylacetamide (60.0 mg, 0.17 mmol) was added to the above solution, which was then stirred for 20 hours at room temperature, and subsequently stirred 20 hours at 60° C. Water was poured onto the reaction mixture, and water layer was extracted with dicholoromethane. Organic extract was dried with anhydrous magnesium sulfate, which was then filtered off, and the filtrate was concentrated under a reduced pressure. The concentrated residue was purified by column chromatography using 10% methanol-ethyl acetate, to obtain 21 mg (32% yield) of desired product. NMR data identifying the product were as follows:

¹H NMR (300 MHz, CDCl₃): δ 7.43 (d, J=14.2 Hz, 2.6 Hz, 1H), 7.05 (dd, J=8.8 Hz, 1.7 Hz, 1H), 6.91 (t, J=9.1 Hz, 1H), 6.40 (t, J=6.3 Hz, 1H), 4.77 (m, 1H), 4.00 (m, 1H), 3.76 (m, 1H), 3.64 (m, 2H), 3.12 (m, 4H), 2.77 (t, J=5.4 Hz, 2H), 2.54 (t, J=5.5 Hz, 2H), 2.00 (s, 3H), 1.93 (s, 3H).

Hydrochloride salt was obtained by treating the product with ethyl ether saturated with hydrogen chloride gas.

### Example 17

Preparation of N-[[(5S)-3-[3-fluoro-4-(4-(2-oxoethylidene)piperidinyl)-phenyl]-2-oxo-5-oxazolidinyl]methyl]acetamide

N-[(5S)-3-[3-fluoro-4-(4-oxopiperid inyl)phenyl]-2-oxo-5-oxazolid inyl]methyl acetamide (100 mg, 0.29 mmol) was dissolved in 3 ml of tetrahydrofuran/water (v/v, 1/3), and indium (39.4 mg, 0.34 mmol) and allyl bromide (37 μl, 0.43 mmol) were added thereto. The resulting solution was stirred for 3 hours and then filtered, and the filtrate was extracted with dichloromethane. Organic extract was dried with magnesium sulfate, which was then filtered off, and the filtrate was concentrated under a reduced pressure, to obtain 96.5 mg (86% yield) of white product.

Preparation of N-[[(5S)-3-[3-fluoro-4-(2,3,4-trihydroxypropylidene)piperidin-yl]phenyl]-2-oxo-5-oxazolidinyl]methylacetamide

N-(5S)-[3-[3-fluoro-4-(4-allyl-4-hydroxypiperidinyl)phenyl]-2-oxo-5-oxazol-idinyl]methylacetamide (20 mg, 0.05 mmol), N-methylmorpholine N-oxide (50% aqueous solution, 19.2 mmol) and catalytic amount of osmium tetraoxide were added to 80% acetone, and the resulting solution was stirred for one hour at room temperature. Magnesium sulfate was added to the above solution, which was then stirred for 10 minutes, and solid was filtered. The filtrate was concentrated under a reduced pressure, to obtain 15.4 mg (68% yield) of yellow solid.

Preparation of N-[(5S)-3-[3-fluoro-4-(1-hydroxy-2-formylpropyl)piperidinyl]-phenyl-2-oxo-5-oxazolidinyl]methylacetamide

N-[(5S)-3-[3-fluoro-4-(1-hydroxy-2,3-dihydroxypropylidene)piperidinyl]-phenyl]-2-oxo-5-oxazolidinyl]methylacetamide (1.38 g, 3.20 mmol) was dissolved in 50% aqueous methanol solution, and sodium periodate (883 mg, 4.13 mmol) was added thereto. The resulting solution was stirred for one and a half hour at room temperature and then extracted with ethyl acetate several times. Organic extracts were collected and dried with anhydrous magnesium sulfate, which was then filtered off, and the filtrate was concentrated under a reduced pressure. The concentrated residue was purified by column chromatography using 10% methanol-ethyl acetate, to obtain 612 mg (49% yield) of desired product.

Preparation of N-[(5S)-3-[3-fluoro-4-(4-(2-oxoethylidene)piperidinyl)-phenyl]-2-oxo-5-oxazolidinyl]methylacetamide and hydrochloride salt thereof

N-[(5S)-3-[3-fl uoro-4-(4-hydroxy-4-(2-formyl)piperidinyl)phenyl]-2-oxo-5-oxazolidinyl]methylacetamide (570 mg, 1.45 mmol) was dissolved in 10 ml of dichloromethane, and triethylamine (505 μl, 3.63 mmol) and 4-N,N-dimethylaminopyridine (354 mg, 2.90 mmol) were added thereto. The resulting solution was stirred for 10 minutes, and methanesulfonyl chloride (224 μl, 2.90 mmol) was slowly added thereto. The resulting solution was then stirred for 3 hours at 0° C. and then washed with water, and water layer was extracted with dichloromethane. Organic extract was dried with anhydrous magnesium sulfate, which was filtered off, and the filtrate was concentrated under a reduced pressure. The concentrated residue was purified by column chromatography, to obtain 120 mg (22% yield) of desired product. NMR data identifying the product as follows.

¹H NMR (CDCl₃, 300 MHz,): δ 10.0 (d, J=8.0 Hz, 1H), 7.45 (dd, J=14.1 Hz, 2.4.Hz, 1H), 7.07 (dd, J=8.7 Hz, 2.3 Hz, 1H), 6.95 (t, J=9.1 Hz, 1H), 6.19 (t, 5.9 Hz, 1H), 5.93 (d, J=8.0Hz, 1H), 4.76 (m, 1H), 4.02 (t, J=8.9 Hz, 1H), 3.76 (t, J=6.7 Hz, 1H), 3.61 (m, 2H), 3.20 (m, 4H), 3.01 (t, J=5.7 Hz, 2H), 2.58 (t, J=5.5 Hz, 2H), 2.01 (s, 3H).

Hydrochloride salt was obtained by treating the product with ethyl ether saturated with hydrogen chloride gas.

### Example 18

Preparation of N-[[(5S)-3-[3-fluoro-4-(4-(2-hydroxyiminoethylidene)-piperidinyl)phenyl]-2-oxo-5-oxazolidinyl]methyl]acetamide and hydrochloride salt thereof

N-[(5S)-3-[3-fluoro-4-(4-(2-oxoethylidene)piperidinyl)phenyl]-2-oxo-5-oxazolidinyl]methylacetamide (30 mg, 0.08 mmol) was dissolved in 1 ml of ethanol/water (v/v, 1/2), and sodium carbonate (5.1 mg, 0.05 mmol) and hydroxylamine hydrochloride salt (7.2 mg, 0.10 mmol) were added thereto. The resulting solution was stirred for 2 hours at 50° C. Water was poured onto the reaction mixture, and water layer was extracted with ethyl acetate. Organic extract was dried with anhydrous magnesium sulfate, which was then filtered off, and the filtrate was concentrated under a reduced pressure, to obtain 22 mg (71 % yield) of desired product. NMR data identifying the product were as follows:

¹H NMR (300 MHz, CD₃OD): δ ppm 8.06 (d, J=10.4 Hz, 1H), 7.46 (dd, J=14.5 Hz, 2.5 Hz, 1H), 7.13 (dd, J=8.8 Hz, 2.3 Hz, 1H), 7.02 (t, J=9.1 Hz, 1H), 5.94 (d, J=10.4 Hz, 1H), 4.77 (m, 1H), 4.09 (t, J=9.1 Hz, 1H), 3.76 (m, 1H), 3.52 (d, J=7.3 Hz, 2H), 3.08 (m, 4H), 2.65 (t, J=5.4 Hz, 1H), 2.59 (t, J=5.5 Hz, 1H), 2.47 (m, 2H), 1.95 (s, 3H).

Hydrochloride salt was obtained by treating the product with ethyl ether saturated with hydrogen chloride gas.

### Example 19

Preparation of N-[[(5S)-3-[3-fluoro-4-(4-(2-methoxyiminoethylidene)-piperidinyl)phenyl]-2-oxo-5-oxazolidinyl]methyl]acetamide and hydrochloride salt thereof

N-[(5S)-3-[3-fluoro-4-(4-(2-oxoethylidene)piperid inyl)phenyl]-2-oxo-5-oxa-zolidinyl]methylacetamide (30 mg, 0.08 mmol) was dissolved in 1 ml of ethanol/water (v/v, 1/2), and sodium carbonate (5.1 mg, 0.05 mmol) and methoxyamine hydrochloride salt (8.7 mg, 0.10 mmol) were added thereto. The resulting solution was stirred for 2 hours at 50° C. Water was poured onto the reaction mixture, and water layer was extracted with ethyl acetate. Organic extract was dried with anhydrous magnesium sulfate, which was then filtered off, and the filtrate was concentrated under a reduced pressure, to obtain 25.7 mg (80% yield) of desired product. NMR data identifying the product were as follows:

¹H NMR (300 MHz, CD₃OD): δ 8.08 (d, J=10.4 Hz, 1H), 7.46 (dd, J=14.3 Hz, 2.3 Hz, 1H), 7.13 (dd, J=8.9 Hz, 2.4 Hz, 1H), 7.02 (t, J=9.2 Hz, 1H), 5.90 (d, J=10.4 Hz, 1H), 4.77 (m, 1H), 4.079 (t, J=9.1 Hz, 1H), 3.80-3.74 (m, 4H), 3.55 (d, J=4.7 Hz, 2H), 3.09 (m, 4H), 2.65 (t, J=5.3 Hz, 1H), 2.59 (t, J=5.2 Hz, 1H), 2.47 (t, J=5.0 Hz, 2H), 2.00 (s, 3H).

Hydrochloride salt was obtained by treating the product with ethyl ether saturated with hydrogen chloride gas.

### Example 20

Preparation of N-[[(5S)-3-[3-fluoro-4-(4-(2-hydroxyiminopropylidene)-piperidinyl)phenyl]-2-oxo-5-oxazolidinyl]methyl]acetamide and hydrochloride salt thereof

N-[(5S)-3-(3-fluoro-4-(4-acetylethylideneperidinyl))-2-oxo-5-oxazolidinyl]-methylacetamide (40 mg, 0.10 mmol) was dissolved in 1 ml of ethanol/water (v/v, 1/2), and sodium carbonate (6.6 mg, 0.06 mmol) and hydroxyamine hydrochloride salt (9.30 mg, 0.13 mmol) were added thereto. The resulting solution was stirred for 2 hours at 50° C. Water was poured onto the reaction mixture, and water layer was extracted with ethyl acetate. Organic extract was dried with anhydrous magnesium sulfate, which was then filtered off, and the filtrate was concentrated under a reduced pressure, to obtain 17.8 mg (43% yield) of desired product. NMR data identifying the product were as follows:

¹H NMR (300 MHz, CDCl₃): δ 7.44 (dd, J=14.6 Hz, 1.1 Hz, 1H), 7.11 (dd, J=8.9 Hz, 1.7 Hz, 1H), 7.04 (m, 1H), 5.70 (s, 1H), 4.75 (m, 1H), 4.08 (t, J=8.9 Hz, 1H), 3.75 (dd, J=9.1 Hz, 6.5 Hz, 1H), 3.52 (m, 2H), 3.18 (t, J=5.5 Hz, 1H), 3.08 (t, J=11.8 Hz, 2H), 2.99 (t, J=5.7 Hz, 2H), 2.69 (t, J=5.5 Hz, 1H), 2.42 (m, 2H), 1.96 (s, 3H).

Hydrochloride salt was obtained by treating the product with ethyl ether saturated with hydrogen chloride gas.

### Example 21

Preparation of N-[[(5S)-3-[3-fluoro-4-(4-(2-methoxyiminopropylidene)-piperidinyl)phenyl]-2-oxo-5-oxazolidinyl]methyl]acetamide and hydrochloride salt thereof

N-[(5S)-3-(3-fluoro-4-(4-acetylethylidenepiperidinyl))-2-oxo-5-oxazolidinyl]-methylacetamide (40 mg, 0.10 mmol) was dissolved in 2 ml of ethanol/water (v/v, 1/1), and potassium carbonate (14.2 mg, 0.10 mmol) and methoxyamine hydrochloride salt (12.9 mg, 0.16 mmol) were added thereto. The resulting solution was stirred for 2 hours at 50° C. Water was poured onto the reaction mixture, and water layer was extracted with ethyl acetate. Organic extract was dried with magnesium sulfate, which was then filtered off, and the filtrate was concentrated under a reduced pressure, to obtain 32.1 mg (74% yield) of desired product. NMR data identifying the product were as follows:

¹H NMR (300 MHz, CDCl₃): δ 7.45 (dd, J=14.5 Hz, 2.3 Hz, 1H), 7.23 (dd, J=5.5 Hz, 2.3 Hz, 1H), 7.04 (m, 1H), 5.65 (s, br, 1H), 4.75 (m, 1H), 4.08 (t, J=9.0 Hz, 1H), 3.76 (m, 4H), 3.55 (d, J=5.0 Hz, 2H), 3.18 (t, J=4.5 Hz, 1H), 3.04 (m, 4H), 2.74 (t, J=4.5 Hz, 2H), 2.42 (t, J=3.0 Hz, 2H), 1.91 (s, 3H).

Hydrochloride salt was obtained by treating the product with ethyl ether saturated with hydrogen chloride gas.

### Example 22

Preparation of N-[[(5S)-3-[3-fluoro-4-(4-(2-hydroxypropylidene)piperidinyl)-phenyl]-2-oxo-5-oxazolidinyl]methyl]acetamide and hydrochloride salt thereof

N-[(5S)-3-(3-fluoro-4-(4-acetylethylidenepiperidinyl))-2-oxo-5-oxazolidinyl]-methylacetamide (25 mg, 0.06 mmol) was dissolved in 2 ml of ethanol/water (v/v, 1/1), and sodium borohydridee (4.8 mg, 0.13 mmol) was added thereto. The resulting solution was stirred for 4 hours at room temperature, to which saturated aqueous ammonium chloride solution was then added, stirred again, and then extracted with dichloromethane. Organic extract was dried with anhydrous magnesium sulfate, which was then filtered off, and the filtrate was concentrated under a reduced pressure, to obtain 19.4 mg (77% yield) of white solid. NMR data identifying the product were as follows:

¹H NMR (300 MHz, CDCl₃): δ 7.39 (dd, J=14.2 Hz, 2.5 Hz, 1H), 7.03 (dd, J=8.8 Hz, 2.3 Hz, 1H), 6.90 (t, J=9.1 Hz, 1H), 6.50 (s, br, 1H), 5.28 (d, J=8.5 Hz, 1H), 4.75 (m, 1H), 4.63 (m, 1H), 3.99 (t, J=9.1 Hz, 1H), 3.75 (m, 1H), 3.63 (m, 2H), 3.02 (m, 4H), 2.47 (m, 2H), 2.34 (m, 2H), 2.00 (s, 3H), 1.29 (d, J=11.3 Hz, 3H).

Hydrochloride salt was obtained by treating the product with ethyl ether saturated with hydrogen chloride gas.

### Example 23

Preparation of N-[[(5S)-3-[3-fluoro-4-(4-(2-acetoxypropylidene)piperidinyl)-phenyl]-2-oxo-5-oxazolidinyl]methyl]acetamide and hydrochloride salt thereof

N-[(5S)-3-[3-fluoro-4-(4-(2-hydroxypropylidene)piperidinyl)phenyl]-2-oxo-5-oxazolidinyl]methylacetamide (30 mg, 0.08 mmol) was dissolved in 1 ml of dichloromethane, and pyridine (11 μl, 0.130 mmol) and purified acetyl chloride (9.2 μl, 0.13 mmol) were slowly added thereto. The resulting solution was stirred for 30 minutes while maintaining temperature at 0° C. The reaction mixture was washed with water, and water layer was extracted with dichloromethane. Organic extract was dried with anhydrous magnesium sulfate, which was then filtered off, and the filtrate was concentrated under a reduced pressure, to obtain 18.8 mg (57% yield) of yellow product. NMR data identifying the product were as follows:

¹H NMR (CDCl₃, 300 MHz,): δ 7.39 (dd, J=14.0 Hz, 2.2 Hz, 1H), 7.03 (dd, J=8.6 Hz, 2.3 Hz, 1H), 6.92 (t, J=9.0 Hz, 1H), 6.41 (t, J=6.1 Hz, 1H), 5.62 (m, 1H), 5.22 (d, J=8.9 Hz, 1H), 4.75 (m, 1H), 4.00 (t, J=9.1 Hz, 1H), 3.73 (m, 1H), 3.63 (m, 2H), 3.04 (m, 4H), 2.54 (m, 1H), 2.45 (m, 1H), 2.34 (m, 2H), 2.03-2.01 (2s, 6H), 1.28 (d, J=6.4 Hz, 3H).

Hydrochloride salt was obtained by treating the product with ethyl ether saturated with hydrogen chloride gas.

### Example 24

Preparation of N-[[(5S)-3-[3-fluoro-4-(4-(2-(chloroacetoxy)propylidene)-piperidinyl)-phenyl]-2-oxo-5-oxazolidinyl]methyl]acetamide and hydrochloride salt thereof

N-[(5S)-3-(3-fluoro-4-(4-(2-hydroxypropylidene)piperidinyl)phenyl)-2-oxo-5-oxazolidinyl]methylacetamide (30 mg, 0.08 mmol) was dissolved in 1 ml of dichloromethane, and pyridine (11 μl, 0.13 mmol) and purified chloroacetyl chloride (10 μl, 0.13 mmol) were slowly added thereto. The resulting solution was stirred for 30 minutes while maintaining the temperature at 0° C. The reaction mixture was washed with water, and water layer was extracted with ethyl acetate. Organic extract was dried with anhydrous magnesium sulfate, which was then filtered off, and the filtrate was concentrated under a reduced pressure, to obtain 25.1 mg (70% yield) of yellow product. NMR data identifying the product were as follows:

¹H NMR (CDCl₃, 300 MHz,): δ 7.39 (dd, J=14.3 Hz, 2.4 Hz, 1H), 7.02 (dd, J=8.9 Hz, 2.3 Hz, 1H), 6.89 (t, J=9.0 Hz, 1H), 5.70 (m, 1H), 5.21 (d, J=9.1 Hz, 1H), 7.45 (m, 1H), 4.02-3.96 (m, 3H), 3.74 (t, J=9.0 Hz, 1H), 3.62 (m, 2H), 3.10-3.06 (m, 2H), 3.06-2.99 (m, 2H), 2.57 (m, 1H), 2.43 (m, 1H), 2.35 (m, 2H), 2.01 (s, 3H), 1.33 (d, J=6.3 Hz, 3H).

Hydrochloride salt was obtained by treating the product with ethyl ether saturated with hydrogen chloride gas.

### Example 25

Preparation of N-[[(5S)-3-[3-fluoro-4-(4-(2-(dichloroacetoxy)propylidene)-piperidinyl)-phenyl]-2-oxo-5-oxazolidinyl]methyl]acetamide and hydrochloride salt thereof

N-[(5S)-3-(3-fluoro-4-(4-(2-hydroxypropylidene)piperidinyl)phenyl)-2-oxo-5-oxazolidinyl]methylacetamide (30 mg, 0.08 mmol) was dissolved in 1 ml of dichloromethane, and pyridine (11 μl, 0.13 mmol) and purified dichloroacetyl chloride (13 μl, 0.13 mmol) were slowly added thereto. The resulting solution was stirred for 30 minutes while maintaining the temperature at 0° C. The reaction mixture was washed with water, and water layer was extracted with ethyl acetate. Organic extract was dried with anhydrous magnesium sulfate, which was then filtered off, and the filtrate was concentrated under a reduced pressure, to obtain 26.4 mg (69% yield) of yellow product. NMR data identifying the product were as follows:

¹H NMR (CDCl₃, 300 MHz,): δ 7.40 (dd, J=14.1 Hz, 2.5 Hz, 1H), 7.02 (dd, J=8.8 Hz, 2.0 Hz, 1H), 6.54 (t, J=5.9 Hz, 1H), 5.90 (s, 1H), 5.72 (m, 1H), 5.26 (d, J=8.9 Hz, 1H), 4.76 (m, 1H), 4.00 (t, J=9.1 Hz, 1H), 3.74 (m, 1H), 3.62 (m, 2H), 3.11 (m, 2H), 2.97 (m, 2H), 2.56 (m, 1H), 2.49-2.32 (m, 3H), 2.02 (s, 3H), 1.38 (d, J=6.4 Hz, 3H).

Hydrochloride salt was obtained by treating the product with ethyl ether saturated with hydrogen chloride gas.

### Example 26

Preparation of N-[[(5S)-3-[3-fluoro-4-(4-(cyanomethylidene)piperidinyl)-phenyl]-2-oxo-5-oxazolidinyl]methyl]thioacetamide and hydrochloride salt thereof

N-[[(5S)-3-[3-fluoro-4-(4-cyanomethylidenepiperidinyl)phenyl]-2-oxo-5-oxa-zolidinyl]methyl]acetamide (30 mg, 0.08 mmol) was dissolved in 2 ml of 1,4-dioxane, and Lawesson's reagent (35 mg, 0.08 mmol) was added thereto. The resulting solution was stirred for 18 hours at 100° C. The reaction mixture was washed with water, and water layer was extracted with dichloromethane. Organic extract was dried with anhydrous magnesium sulfate, which was then filtered off, and the filtrate was concentrated under a reduced pressure. The concentrated residue was purified by column chromatography using 10% methanol-ethyl acetate, to obtain 18.3 mg (52% yield) of desired product. NMR data identifying the product were as follows:

¹H NMR (CDCl₃, 300 MHz,): δ 8.39 (s, br, 1H), 7.45 (d, J=13.5 Hz, 1H), 7.05 (s, br, 2H), 5.21 (s, 1H), 4.99 (m, 1H), 4.21-4.18 (m, 1H), 4.13-4.04 (m, 2H), 3.84 (t, J=9.2 Hz, 1H), 3.23-3.16 (m, 4H), 2.81 (t, J=5.4 Hz, 2H), 2.59 (s, 5H).

Hydrochloride salt was obtained by treating the product with ethyl ether saturated with hydrogen chloride gas.

### Example 27

Anti-microbial activity test in vitro

Strains were cultivated for 18 hours at 37° C. in Agar Dilution method using Mueller Hinton Agar, and then plane plates inoculated with the strains by diluting them two times gradually were aligned in a row. Minimum inhibition concentration (MIC, μg/ml) for the representative compounds of the present invention, Linezolid (LZD) and vancomycin (VAN) were decided through visual observation, and the results are shown in the following tables 2 and 3.

TABLE 2

| | Compd. Nos. | | | | | | | | | | | |
|---|---|---|---|---|---|---|---|---|---|---|---|---|
| Strain | 6 | 7 | 8 | 9 | 10 | 11 | 12 | 13 | 14 | 15 | 16 | 17 |
| S. aureus | 2 | 4 | 2 | 32 | 16 | 16 | 8 | 16 | 8 | 32 | 2 | 8 |
| ATCC | | | | | | | | | | | | |
| 29213 | | | | | | | | | | | | |
| MRSA | 4 | 4 | 2 | 32 | 16 | 8 | 8 | 16 | 8 | 16 | 2 | 4 |
| C2207 | | | | | | | | | | | | |
| MRSA | 4 | 4 | 2 | 16 | 8 | 8 | 8 | 8 | 4 | 16 | 2 | 4 |
| C5100 | | | | | | | | | | | | |
| MRSA | 2 | 4 | 1 | 16 | 8 | 8 | 16 | 8 | 4 | 16 | 2 | 4 |
| C6068 | | | | | | | | | | | | |
| CRSA | 4 | 4 | 2 | 16 | 16 | 8 | 16 | 8 | 8 | 16 | 2 | 4 |
| C6043 | | | | | | | | | | | | |
| CRSA | 4 | 4 | 2 | 32 | 16 | 8 | 8 | 16 | 16 | 16 | 2 | 8 |
| C1062 | | | | | | | | | | | | |
| MSSA | 4 | 4 | 2 | 32 | 8 | 16 | 16 | 8 | 8 | 8 | 2 | 4 |
| C7142 | | | | | | | | | | | | |
| MSSA | 4 | 4 | 4 | 16 | 16 | 16 | 16 | 16 | 8 | 8 | 2 | 8 |
| C2214 | | | | | | | | | | | | |
| S. epidermis | 1 | 1 | 0.5 | 4 | 2 | 2 | 1 | 4 | 1 | 2 | 0.5 | 1 |
| ATCC12228 | | | | | | | | | | | | |
| S. epidermis | 2 | 2 | 2 | 8 | 4 | 8 | 8 | 16 | 4 | 4 | 2 | 2 |
| C2230 | | | | | | | | | | | | |
| S. epidermis | 2 | 2 | 1 | 8 | 4 | 4 | 8 | 8 | 1 | 4 | 2 | 2 |
| C2235 | | | | | | | | | | | | |
| E. faecalis | 4 | 4 | 2 | 16 | 16 | 8 | 16 | 16 | 8 | 8 | 2 | 16 |
| ATCC29212 | | | | | | | | | | | | |
| E. faecalis | 4 | 4 | 2 | 16 | 16 | 8 | 16 | 16 | 4 | 8 | 2 | 4 |
| C6288 | | | | | | | | | | | | |
| E. faecalis | 4 | 4 | 2 | 16 | 8 | 8 | 16 | 8 | 8 | 4 | 2 | 8 |
| C6291 | | | | | | | | | | | | |
| E. faecium | 4 | 4 | 2 | 16 | 8 | 8 | 8 | 8 | 8 | 4 | 2 | 8 |
| C2252 | | | | | | | | | | | | |
| E. faecium | 2 | 2 | 2 | 16 | 8 | 8 | 8 | 8 | 4 | 2 | 2 | 4 |
| C6301 | | | | | | | | | | | | |
| S. pyogenes | 1 | 1 | 0.5 | 2 | 2 | 2 | 8 | 8 | 4 | 2 | 2 | 2 |
| ATCC8668 | | | | | | | | | | | | |
| S. pyogenes | 4 | 4 | 2 | 16 | 8 | 8 | 0.2 | 4 | 2 | 2 | 0.5 | 8 |
| C6003 | | | | | | | (5) | | | | | |
| S. pyogenes | 1 | 1 | 0.5 | 4 | 2 | 2 | 16 | 16 | 8 | 8 | 2 | 1 |
| C6012 | | | | | | | | | | | | |
| VRE C6487 | 4 | 2 | 4 | 16 | 4 | 8 | 2 | 1 | 1 | 2 | 0.5 | 8 |
| VRE C6488 | 2 | 2 | 4 | 16 | 8 | 8 | 8 | 8 | 8 | 4 | 1 | 8 |

TABLE 3

| | Compd. Nos. | | | | | | | | | | | |
|---|---|---|---|---|---|---|---|---|---|---|---|---|
| Strain | 18 | 19 | 20 | 21 | 22 | 23 | 24 | 25 | 26 | 27 | LZD | VAN |
| S. aureus | 8 | 8 | 8 | 16 | 8 | 16 | 16 | 16 | 2 | 2 | 4 | 1 |
| ATCC29213 | | | | | | | | | | | | |
| MRSA C2207 | 8 | 4 | 8 | 16 | 16 | 8 | 16 | 16 | 2 | 2 | 4 | 1 |
| MRSA C5100 | 4 | 4 | 8 | 8 | 8 | 16 | 16 | 8 | 1 | 1 | 2 | 2 |
| MRSA C6068 | 4 | 4 | 4 | 8 | 4 | 8 | 8 | 8 | 2 | 2 | 2 | 1 |
| CRSA C6043 | 8 | 4 | 8 | 16 | 8 | 16 | 16 | 16 | 2 | 2 | 2 | 2 |
| CRSA C1062 | 8 | 4 | 8 | 32 | 16 | 16 | 8 | 16 | 2 | 2 | 4 | 2 |
| MSSA C7142 | 8 | 4 | 16 | 16 | 16 | 16 | 16 | 16 | 2 | 2 | 4 | 2 |
| MSSA C2214 | 8 | 8 | 8 | 16 | 16 | 16 | 16 | 16 | 2 | 2 | 4 | 1 |
| S. epidermidis | 1 | 1 | 1 | 2 | 2 | 2 | 2 | 2 | 0.5 | 0.5 | 0.5 | 1 |
| ATCC12228 | | | | | | | | | | | | |
| S. epidermidis | 4 | 4 | 4 | 16 | 4 | 8 | 8 | 8 | 1 | 1 | 2 | 2 |
| C2230 | | | | | | | | | | | | |
| S. epidermidis | 4 | 2 | 4 | 8 | 4 | 8 | 8 | 8 | 0.5 | 0.5 | 2 | 2 |
| C2235 | | | | | | | | | | | | |
| E. faecalis | 8 | 4 | 8 | 16 | 8 | 8 | 8 | 8 | 2 | 2 | 4 | 4 |
| ATCC29212 | | | | | | | | | | | | |
| E. faecalis | 4 | 2 | 4 | 8 | 4 | 8 | 16 | 8 | 1 | 1 | 2 | 2 |
| C6288 | | | | | | | | | | | | |
| E. faecalis | 4 | 4 | 4 | 8 | 8 | 8 | 16 | 8 | 1 | 1 | 2 | 2 |
| C6291 | | | | | | | | | | | | |
| E. faecium | 4 | 4 | 4 | 8 | 4 | 8 | 16 | 8 | 1 | 1 | 2 | 1 |
| C2252 | | | | | | | | | | | | |
| E. faecium | 4 | 4 | 4 | 8 | 4 | 8 | 8 | 8 | 1 | 1 | 2 | 1 |
| C6301 | | | | | | | | | | | | |
| S. pyogenes | 2 | 1 | 1 | 2 | 2 | 0.5 | 2 | 2 | 0.5 | 0.5 | 0.5 | 0.12 |
| ATCC8668 | | | | | | | | | | | | |
| S. pyogenes | 4 | 2 | 4 | 8 | 8 | 8 | 8 | 8 | 0.25 | 0.25 | 2 | 4 |
| C6003 | | | | | | | | | | | | |
| S. pyogenes | 1 | 1 | 1 | 2 | 2 | 2 | 2 | 2 | 0.25 | 0.25 | 0.5 | 0.5 |
| C6012 | | | | | | | | | | | | |
| VRE C6487 | 4 | 4 | 4 | 4 | 4 | 4 | 4 | 4 | 0.5 | 0.5 | 2 | >32 |
| VRE C6488 | 4 | 4 | 4 | 4 | 4 | 4 | 4 | 4 | 0.5 | 0.5 | 2 | >32 |

According to the present invention an oxazolidinone compound or a salt thereof, and a preparation method thereof was provided as described above. As shown in tables 2 and 3, it was discovered in vitro experiments that the compound of the present invention shows superior biological activities against the strains exhibiting resistance to the conventional antibiotics. Therefore, the compound according to the present invention is expected to be used to treat infectious diseases caused by viruses exhibiting resistance to the conventional antibiotics.

As the present invention may be embodied in several forms without departing from the spirit or essential characteristics thereof, it should also be understood that the above-described embodiments are not limited by any of the details of the foregoing description, unless otherwise specified, but, rather should be construed broadly within its spirit and scope as defined in the appended claims, and therefore all changes and modifications that fall within the metes and bounds of the claims, or equivalence of such metes and bounds are therefore intended to be embraced by the appended claims.

What is claimed is:

1. A methylidene oxazolidinone compound represented by the following formula (1) or a pharmaceutically acceptable salt thereof: 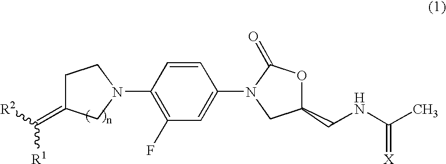
wherein X represents an oxygen or sulfur atom;
R¹ and R² independently represent hydrogen atom, cyano group, alkyl group, halogen atom, acetoxy group, ethoxycarbonyl group, hydroxy group, hydroxyimino group, methoxyimino group or aminoethyl group, or a unsaturated 5-membered heterocyclic substituent containing one or more hetero atoms selected from the group consisting of oxygen, nitrogen and sulfur; and
n represents an integer 1 or 2.

2. The compound according to claim 1, wherein the alkyl group is methyl, ethyl or propyl group, the halogen atom is chlorine or bromine atom, the acetoxy group is the one substituted with one or more chlorine atoms, and the 5-membered heterocyclic substituent is isoxazole, thiophene, thiazole, isothiazole or thiadiazole.

3. The compound according to claim 1, which is N-[[(5S)-3-[3-fluoro-4-(3-dicyanomethylidenepyrrolidin-1-yl)phenyl]-2-oxo-5-oxazolidinyl]methyl]acetamide, N-[[(5S)-3-[3-fluoro-4-((3-(1-ethoxycarbonyl-1-cyano)methylidene)pyrrolidin-1-yl)-phenyl]-2-oxo-5-oxazolidinyl]methyl]acetamide, N-[[(5S)-3-[3-fluoro-4-(3-cyano-methylidenepyrrolidin-1-yl)-phenyl]-2-oxo-5-oxazolidinyl]methyl]acetamide, N-[[(5S)-3-[3-fluoro-4-((3-(1-methyl-1-cyano)methylidene)pyrrolidin-1-yl)phenyl]-2-oxo-5-oxazolidinyl]methyl]acetamide, N-[[(5S)-3-[3-fluoro-4-(4-(1-cya no-2-ethoxy-carbonylethylidene)piperidin-1-yl)phenyl]-2-oxo-5-oxazolidinyl]methyl]acetamide, N-[(5S)-3-[3-fluoro-4-(4-dicyanomethylidenepyrrolidin-1-yl)phenyl]-2-oxo-5-oxazol-idinyl]methylacetamide, N-[[(5S)-3-[3-fluoro-4-((4-(1-ethoxycarbonyl-1-cyano)-methylidene)piperidinyl)phenyl]-2-oxo-5-oxazolidinyl]methyl]acetamide, N-[[(5S)-3-[3-fluoro-4-(4-cyanomethylidenepiperidinyl)-phenyl]-2-oxo-5-oxazolidinyl]methyl]-acetamide, N-[[(5S)-3-[3-fluoro-4-((4-(3-thiophen-2-yl-5-isoxazolyl)methylidene)-piperidinyl)phenyl]-2-oxo-5-oxazolidinyl]methyl]acetamide, N-[[(5S)-3-[3-fluoro-4-((4-(3-(3-methyl-isothiazol-4-yl)-iso-xazolyl)methylidene)piperidinyl)phenyl]-2-oxo-5-oxazolidinyl]methyl]acetamide, N-[[(5S)-3-[3-fluoro-4-((4-ethoxycarbonyl-methylidene)piperidinyl)phenyl]-2-oxo-5-oxazolidinyl]methyl]acetamide, N-[[(5S)-3-[3-fluoro-4-(4-methylcarbonylmethylidene-piperidinyl)phenyl]-2-oxo-5-oxazolidinyl]-methyl]acetamide, N-[[(5S)-3-[3-fluoro-4-(4-(1-ethoxycarbonylethylidene)-piperidinyl)phenyl]-2-oxo-5-oxazolidinyl]methyl]acetamide, N-[[(5S)-3-[3-fluoro-4-(4-carboxymethylidenepiperidin yl)-phenyl]-2-oxo-5-oxazolidinyl]methyl]acetamide, -[[(5S)-3-[3-fluoro-4-((4-(1-ethoxycarbonyl-1-chloro)methylidene)piperidinyl)-phenyl]-2-oxo-5-oxazolidinyl]methyl]acetamide, N-[[(5S)-3-[3-fluoro-4-(4-(1-cyanoethylidene)piperidinyl)phenyl]-2-oxo-5-oxazolid inyl]methyl]acetamide, N-[[(5S)-3-[3-fluoro-4-(4-(2-oxoethylidene)piperidinyl)-phenyl]-2-oxo-5-oxazolidinyl]-methyl]acetamide, N-[[(5S)-3-[3-fluoro-4-(4-(2-hydroxyiminoethylidene)piperidinyl)-phenyl]-2-oxo-5-oxazolidinyl]methyl]acetamide, N-[[(5S)-3-[3-fluoro-4-(4-(2-methoxyiminoethylidene)piperidinyl)phenyl]-2-oxo-5-oxazolidinyl]methyl]acetamide, N-[[(5S)-3-[3-fluoro-4-(4-(2-hydroxyiminopropylidene)piperidinyl)phenyl]-2-oxo-5-oxazolidinyl]methyl]acetamide, N-[[(5S)-3-[3-fluoro-4-(4-(2-methoxyimino-propyl-idene)piperidinyl)phenyl]-2-oxo-5-oxazolidinyl]methyl]acetamide, N-[[(5S)-3-[3-fluoro-4-(4-(2-hydroxypropylidene)piperidinyl)phenyl]-2-oxo-5-oxazolidinyl]methyl]-acetamide, N-[[(5S)-3-[3-fluoro-4-(4-(2-acetoxypropylidene)piperidinyl)- phenyl]-2-oxo-5-oxazolidinyl]methyl]acetam ide, N-[[(5S)-3-[3-fluoro-4-(4-(2-(chloroacetoxy)-propylidene)piperidinyl)-phenyl]-2-oxo-5-oxazolidinyl]methyl]-acetamide, N-[[(5S)-3-[3-fluoro-4-(4-(2-(dichloroacetoxy)propylidene)piperidinyl)-phenyl]-2-oxo-5-oxazolidinyl]methyl]acetamide, N-[[(5S)-3-[3-fluoro-4-(4-(cyano-methylidene)-piperidinyl)phenyl]-2-oxo-5-oxazolidinyl]methyl]thioacetamide, or a hydro-chloride salt thereof.

4. The compound according to claim 1, wherein the pharmaceutically acceptable salt is a methanesulfonate, fumarate, hydrobromide salt, citrate, maleate, phosphate, sulfate, hydrochloride salt or a sodium salt.

5. A method for preparing a compound of formula (1) which comprises reacting a compound of formula (2) with a compound of formula (3) in the presence of a catalyst, using or without using a solvent: 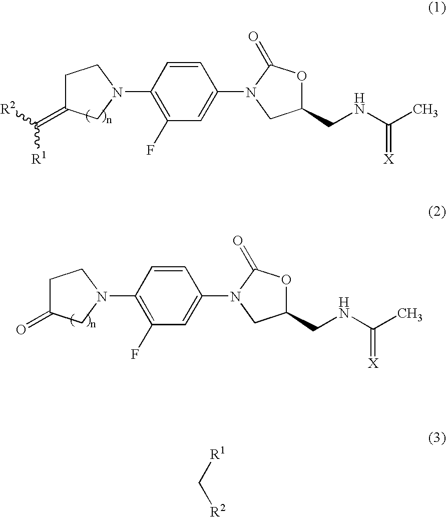
wherein X represents an oxygen or sulfur atom;
R¹ and R² independently represent hydrogen atom, cyano group, alkyl group, halogen atom, acetoxy group, ethoxycarbonyl group, hydroxy group, hydroxyimino group, methoxyimino group or.aminoethyl group, or a unsaturated 5-membered heterocyclic substituent containing one or more hetero atoms selected from the group consisting of oxygen, nitrogen and sulfur; and
n represents an integer 1 or 2.

6. The method according to claim 5, wherein R¹ is cyano group and R² is cyano or ethoxycarbonyl group.

7. The method according to claim 5, wherein the solvent is dichloromethane or benzene.

8. The method according to claim 5, wherein the catalyst is selected from the group consisting of alumina, ammonia, triethylamine, pyridine, piperidine, potassium fluoride, cerium fluoride and titanium chloride.

9. The method according to claim 5, wherein the reaction is carried out at room temperature or at 50-100° C.

10. A method for preparing a compound of formula (1) which comprises reacting a compound of formula (2) with a compound of formula (4) using a base and a solvent: 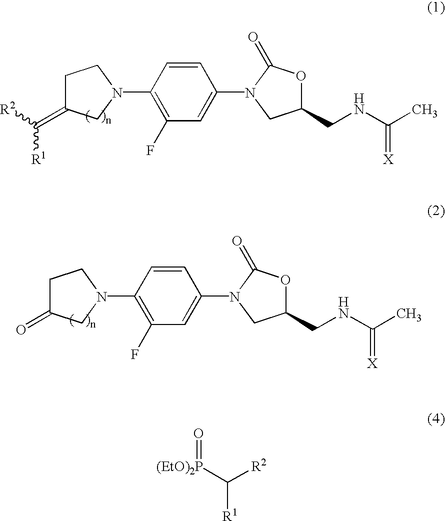
wherein X represents an oxygen or sulfur atom;
R¹ and R² independently represent hydrogen atom, cyano group, alkyl group, halogen atom, acetoxy group, ethoxycarbonyl group, hydroxy group, hydroxyimino group, methoxyimino group or aminoethyl group, or a unsaturated 5-membered heterocyclic substituent containing one or more hetero atoms selected from the group consisting of oxygen, nitrogen and sulfur; and
n represents an integer 1 or 2.

11. The method according to claim 10, wherein the solvent is selected from the group consisting of tetrahydrofuran, dimethylethane and dimethylformamide.

12. The method according to claim 10, wherein the base is sodium hydride or potassium t-butoxide.

13. The method according to claim 10, wherein the reaction is carried out at room temperature or at 40-100° C.
